(19) United States
(12) Patent Application Publication
(10) Pub. No.: US 2007/0111889 A1
(43) Pub. Date: May 17, 2007

(54) 6-(Aminocarbonyl-phenyl)triazolopyrimidines, methods for the production thereof, use thereof for controlling harmful fungi, and substances containing the same

(75) Inventors: Jordi Tormo i Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagne, Neustadt (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 10/580,990

(22) PCT Filed: Dec. 17, 2004
(86) PCT No.: PCT/EP04/14393

§ 371 Date: May 26, 2006

(30) Foreign Application Priority Data
Dec. 19, 2003 (DE) 10360392.1
Jan. 23, 2004 (DE) 102004003767.1
Apr. 19, 2004 (DE) 102004019456.4

(51) Int. Cl. A01N 43/90; C07D 487/04

(52) U.S. Cl. 504/100; 514/259.31; 544/263

(57) ABSTRACT

Triazolopyrimidines of the formula I 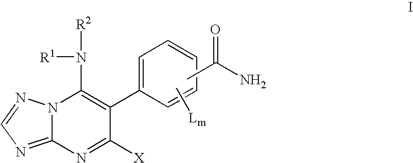 in which the substituents are as defined below:
R¹, R² are hydrogen, alkyl, haloalkyl, cycloalkyl, halocycloalkyl, alkenyl, haloalkenyl, cycloalkenyl, halocycloalkenyl, alkynyl, haloalkynyl or phenyl, naphthyl or a five- or six-membered saturated, partially unsaturated or aromatic heterocycle which contains one to four heteroatoms from the group consisting of O, N and S,
   R¹ and R² together with the nitrogen atom to which they are attached may also form a five- or six-membered heterocyclyl or heteroaryl which is attached via N and may contain a further heteroatom from the group consisting of O, N and S as ring member and may be substituted as defined in the description; L is halogen, cyano, alkyl, haloalkyl, alkoxy, alkenyloxy or alkoxycarbonyl; m is 1, 2, 3 or 4, where the groups L may be different if m is greater than 1; X is halogen, cyano, alkyl, haloalkyl, alkoxy or haloalkoxy; Processes and intermediates for preparing these compounds, compositions comprising them and their use for controlling phytopathogenic harmful fungi

The present invention relates to substituted triazolopyrimidines of the formula I 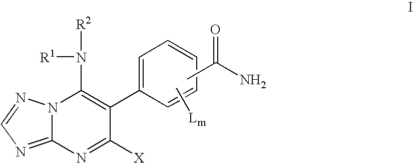
in which the substituents are as defined below:
R¹, R² independently of one another are hydrogen, C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl or a five- or six-membered saturated, partially unsaturated or aromatic heterocycle which contains one to four heteroatoms from the group consisting of O, N and S,
   R¹ and R² together with the nitrogen atom to which they are attached may also form a five- or six-membered heterocyclyl or heteroaryl which is attached via N and may contain one to three further heteroatoms from the group consisting of O, N and S as ring members and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
R¹ and/or R² may carry one to four identical or different groups R^{a}:
   R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkylenoxy, phenyl, naphthyl, a five- to ten-membered saturated, partially unsaturated or aromatic heterocycle which contains one to four heteroatoms from the group consisting of O, N and S,
   where these aliphatic, alicyclic or aromatic groups for their part may be partially or fully halogenated or may carry one to three groups R^{b}:
   R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the abovementioned alkenyl or alkynyl groups in these radicals contain 2 to 8 carbon atoms;
      and/or one to three of the following radicals:
      cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;
L is halogen, cyano, C₁-C₆-alkyl, C₁-C₄-haloalkyl, C₁-C₆-alkoxy, C₃-C₆-alkenyloxy or C₁-C₄-alkoxycarbonyl;
m is 1, 2, 3 or 4, where the groups L may be different if m is greater than 1;
X is halogen, cyano, C₁-C₄-alkyl, C₁-C₄-haloalkyl, C₁-C₄-alkoxy or C₁-C₂-haloalkoxy.

Moreover, the invention relates to processes and intermediates for preparing these compounds, to compositions comprising them and to their use for controlling phytopathogenic harmful fungi.

5-Chloro-6-phenyl-7-aminotriazolopyrimidines are known in a general manner from EP-A 71 792 and EP-A 550 113. 6-Phenyltriazolopyrimidines whose phenyl group may carry an alkylamide group in the para-position are proposed in a general manner in WO 03/080615. These compounds are known to be suitable for controlling harmful fungi.

The compounds according to the invention differ from those described in WO 03/080615 by the carboxamide group as substituent of the 6-phenyl ring.

However, the action of the known compounds is in many cases unsatisfactory. On the basis thereof, it is an object of the present invention to provide compounds having improved activity and/or a broader activity spectrum.

We have found that this object is achieved by the compounds defined at the outset. Moreover, we have found processes and intermediates for their preparation, compositions comprising them and methods for controlling harmful fungi using the compounds I.

The compounds according to the invention can be obtained by different routes. Advantageously, they are prepared by reacting 5-aminotriazol of the formula II with appropriately substituted phenylmalonates of the formula III in which R is alkyl, preferably C₁-C₆-alkyl, in particular methyl or ethyl.

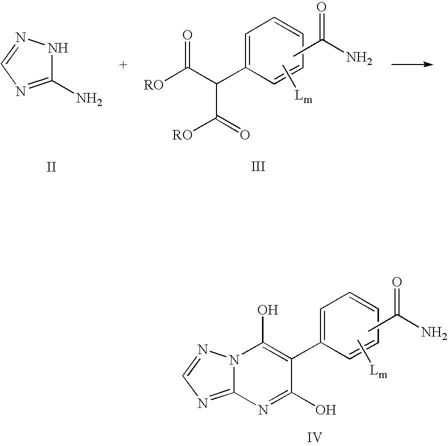

This reaction is usually carried out at temperatures of from 80° C. to 250° C., preferably from 120° C. to 180° C., without solvent or in an inert organic solvent in the presence of a base [cf. EP-A 770 615] or in the presence of acetic acid under the conditions known from Adv. Het. Chem. 57 (1993), 81 ff.

Suitable solvents are aliphatic hydrocarbons, aromatic hydrocarbons, such as toluene, o-, m- and p-xylene, halogenated hydrocarbons, ethers, nitrites, ketones, alcohols, and also N-methylpyrrolidone, dimethyl sulfoxide, dimethylformamide and dimethylacetamide. With particular preference, the reaction is carried out in the absence of a solvent or in chlorobenzene, xylene, dimethyl sulfoxide or N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, organometallic compounds, in particular alkali metal alkyls, alkyl magnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines, such as triisopropylethylamine, tributylamine, N-methylmorpholine or N-methylpiperidine.

The bases are generally employed in catalytic amounts; however, they can also be employed in equimolar amounts, in excess or, if appropriate, as solvent.

The starting materials are generally reacted with one another in equimolar amounts. In terms of yield, it may be advantageous to use an excess of the base and the malonate III, based on the triazole.

Phenylmalonates of the formula III are advantageously obtained from the reaction of appropriately substituted bromobenzenes with dialkyl malonates under Cu(I) catalysis [cf. Chemistry Letters (1981), 367-370; EP-A 10 02 788].

The dihydroxytriazolopyrimidines of the formula IV are, under the conditions known from WO-A 94/20501, converted into the dihalopyrimidines of the formula V in which Hal is a halogen atom, preferably a bromine or a chlorine atom, in particular a chlorine atom. The halogenating agent [HAL] used is advantageously a chlorinating agent or a brominating agent, such as phosphorus oxybromide or phosphorus oxychloride, if appropriate in the presence of a solvent.

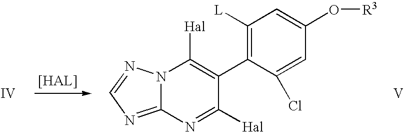

This reaction is usually carried out at from 0° C. to 150° C., preferably from 80° C. to 125° C. [cf. EP-A 770 615].

Dihalopyrimidines of the formula V are reacted further with amines of the formula VI 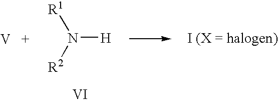
in which R¹ and R² are as defined in formula I, to give compounds of the formula I in which X is halogen.

This reaction is advantageously carried out at from 0° C. to 70° C., preferably from 10° C. to 35° C., preferably in the presence of an invert solvent, such as an ether, for example dioxane, diethyl ether or, in particular, tetrahydrofuran, a halogenated hydrocarbon, such as dichloromethane, or an aromatic hydrocarbon, such as, for example, toluene [cf. WO-A 98/46608].

The use of a base, such as a tertiary amine, for example triethylamine, or an inorganic base, such as potassium carbonate, is preferred; it is also possible for excess amine of the formula VI to serve as base.

Compounds of the formula I in which X is cyano, C₁-C₆-alkoxy or C₁-C₂-haloalkoxy can be obtained in an advantageous manner by reacting compounds I in which X is halogen, preferably chlorine, with compounds M-X′ (formula VII). Depending on the meaning of the group X′ to be introduced, the compounds VII are inorganic cyanides, alkoxides or haloalkoxides. The reaction is advantageously carried out in the presence of an inert solvent. The cation M in formula VII is of little importance; for practical reasons, ammonium, tetraalkylammonium or alkali metal or alkaline earth metal salts are usually preferred.

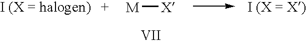

The reaction temperature is usually from 0 to 120° C., preferably from 10 to 40° C. [cf. J. Heterocycl. Chem. 12 (1975), 861-863].

Suitable solvents include ethers, such as dioxane, diethyl ether and, preferably, tetrahydrofuran, halogenated hydrocarbons, such as dichloromethane, and aromatic hydrocarbons, such as toluene.

Compounds of the formula I, in which X is C₁-C₄-alkyl or C₁-C₄-haloalkyl can be obtained in an advantageous manner by the following synthesis route:

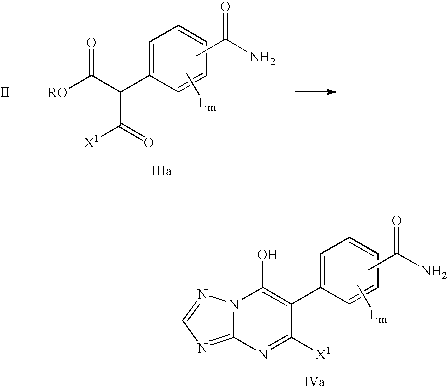

Starting with the keto esters IIIa, the 5-alkyl-7-hydroxy-6-phenyltriazolopyrimidines IVa are obtained. In the formula IIIa and IVa, X¹ is C₁-C₄-alkyl or C₁-C₄-haloalkyl. By using the easily obtainable 2-phenylacetoacetates (IIIa where X¹=CH₃), the 5-methyl-7-hydroxy-6-phenyltriazolopyrimidines are obtained [cf. Chem. Pharm. Bull. 9 (1961), 801]. The starting materials Ilia are advantageously prepared under the conditions described in EP-A 10 02 788.

The resulting 5-alkyl-7-hydroxy-6-phenyltriazolopyrimidines are reacted with halogenating agents [HAL] under the conditions described further above to give the 7-halotriazolopyrimidines of the formula Va. Preference is given to using chlorinating or brominating agents, such as phosphorus oxybromide, phosphorus oxychloride, thionyl chloride, thionyl bromide or sulfuryl chloride. The reaction can be carried out neat or in the presence of a solvent. Customary reaction temperatures are from 0 to 150° C. or, preferably, from 80 to 125° C.

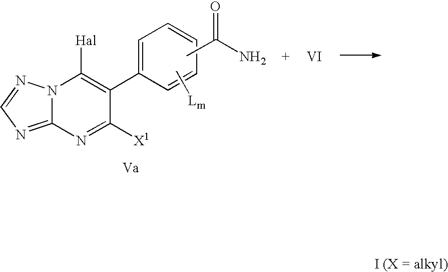

The reaction of Va with amines VI is carried out under the conditions described further above.

Alternatively, compounds of the formula I in which X is C₁-C₄-alkyl can also be prepared from compounds I in which X is halogen, in particular chlorine, and malonates of the formula VIII. In formula VIII, X″ is hydrogen or C₁-C₃-alkyl and R is C₁-C₄-alkyl. They are converted into compounds of the formula IX and decarboxylated to give compounds I [cf. U.S. Pat. No. 5,994,360].

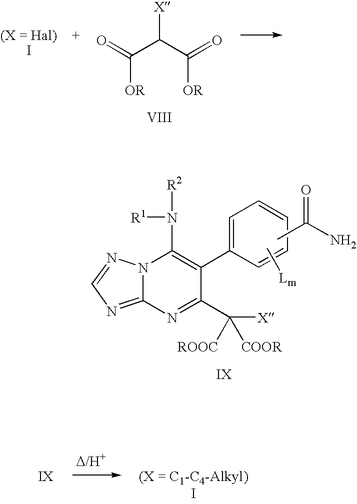

The malonates VIII are known from the literature [J. Am. Chem. Soc. 64 (1942), 2714; J. Org. Chem. 39 (1974), 2172; Helv. Chim. Acta, 61 (1978), 1565], or they can be prepared in accordance with the literature cited.

The subsequent hydrolysis of the ester IX is carried out under generally customary conditions; depending on the various structural elements, the alkaline or the acidic hydrolysis of the compounds IX may be advantageous. Under the conditions of the ester hydrolysis, there may already be complete or partial decarboxylation to I.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particularly preferably, the reaction is carried out in hydrochloric acid or acetic acid. It is also possible to use mixtures of the solvents mentioned.

Compounds of the formula I in which X is C₁-C₄-alkyl can also be obtained by coupling 5-halotriazolopyrimidines of the formula I in which X is halogen with organometallic reagents of the formula X. In one embodiment of this process, the reaction is carried out with transition metal catalysis, such as Ni or Pd catalysis.

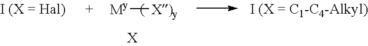

In formula X, M is a metal ion of the valency y, such as, for example, B, Zn or Sn, and X″ is C₁-C₃-alkyl. This reaction can be carried out, for example, analogously to the following methods: J. Chem. Soc. Perkin Trans. 1 (1994), 1187, ibid 1 (1996), 2345; WO-A 99/41255; Aust. J. Chem. 43 (1990), 733; J. Org. Chem. 43 (1978), 358; J. Chem. Soc. Chem. Commun. (1979), 866; Tetrahedron Lett. 34 (1993), 8267; ibid 33 (1992), 413.

Alternatively, compounds of the formula I can also be prepared from substituted 6-cyanophenyltriazolopyrimidines of the formula XI.

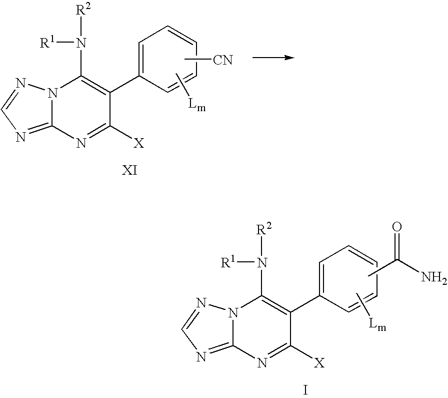

This reaction can be carried out in the presence of sulfuric acid at from 0 to 60° C., in particular from 20 to 25° C. [cf. Synthetic Commun. (1999), 547 ff.], alternatively, the reaction can be carried out in the system polyethylene glycol/NaOH at from 0 to 60° C., in particular from 20 to 25° C., in the presence of a solvent or diluent [cf. Synthetic Commun. (2000), 1713 ff.], or using urea/hydrogen peroxide at from 0 to 60° C., in particular from 20 to 25° C., in the presence of a solvent or diluent [cf. Org. Lett. (1999), 189 ff.].

Compounds of the formula XI are known from WO 03/080615 and/or can be prepared in accordance with the literature cited.

Moreover, the compounds of the formula I can be prepared by Pd-catalyzed aminocarbonylation [cf. Tetrahedron Lett. 39 (1988), 2835-2838; J. Org. Chem. 66, (2001), 4311 ff; WO 00/37428; DE 35 25 564; J. Org. Chem. 62 (1997), 8640-8653; JP 2000191612) of the corresponding halogen compounds XII [cf. EP-A 550113] or triflate XIII.

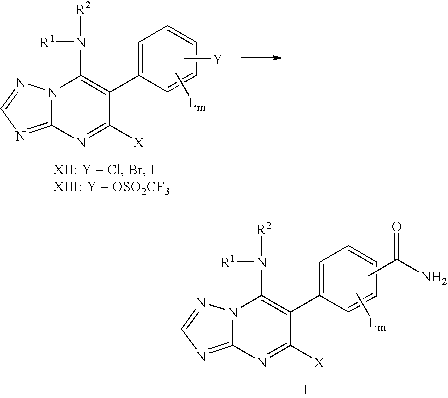

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of the symbols given in the formulae above, collective terms were used which are generally representative of the following substituents:
halogen: fluorine, chlorine, bromine and iodine;
alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated straight-chain or branched hydrocarbon radicals having 2 to 8 carbon atoms and one or two double bonds in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
five- to ten-membered saturated, partially unsaturated or aromatic heterocycle which contains one to four heteroatoms from the group consisting of O, N and S:
   5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
   5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
   6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;
oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The scope of the present invention includes the (R) and (S)-isomers and the racemates of compounds of the formula I having chiral centers.

The particularly preferred embodiments of the intermediates with respect to the variables correspond to those of radicals L and R³ of formula I.

With a view to the intended use of the triazolopyrimidines of the formula I, particular preference is given to the following meanings of the substituents, in each case on their own or in combination:

Preference is given to compounds of the formula I in which R¹ is not hydrogen.

Particular preference is given to compounds I in which R¹ is C₁-C₆-alkyl, C₂-C₆-alkenyl or C₁-C₈-haloalkyl.

Preference is given to compounds I in which R¹ is a group A: 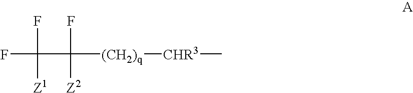
in which
Z¹ is hydrogen, fluorine or C₁-C₆-fluoroalkyl,
Z² is hydrogen or fluorine, or
Z¹ and Z² together form a double bond;
q is 0 or 1; and
R³ is hydrogen or methyl.

Moreover, preference is given to compounds I in which R¹ is C₃-C₆-cycloalkyl which may be substituted by C₁-C₄-alkyl.

Particular preference is given to compounds I in which R² is hydrogen.

Preference is likewise given to compounds I in which R² is methyl or ethyl.

If R¹ and/or R² comprise haloalkyl or haloalkenyl groups having a center of chirality, the (S) isomers are preferred for these groups. In the case of halogen-free alkyl or alkenyl groups having a center of chirality in R¹ or R², preference is given to the (R) configured isomers.

Preference is furthermore given to compounds I in which R¹ and R² together with the nitrogen atom to which they are attached form a piperidinyl, morpholinyl or thiomorpholinyl ring, in particular a piperidinyl ring, which, if appropriate, is substituted by one to three groups halogen, C₁-C₄-alkyl or C₁-C₄-haloalkyl. Particularly preference is given to the compounds in which R¹ and R² together with the nitrogen atom to which they are attached form a 4-methylpiperidine ring.

The invention furthermore preferably provides compounds I in which R¹ and R² together with the nitrogen atom to which they are attached form a pyrazole ring which, if appropriate, is substituted by one or two groups halogen, C₁-C₄-alkyl or C₁-C₄-haloalkyl, in particular by 3,5-dimethyl or 3,5-di(trifluoromethyl).

In addition, particular preference is also given to compounds of the formula I in which R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; R² is hydrogen or methyl; or R¹ and R² together are —(CH₂)₂CH(CH₃)(CH₂)₂—, —(CH₂)₂CH(CF₃)(CH₂)₂— or —(CH₂)₂O(CH₂)₂—.

Preference is given to compounds I in which X is halogen, C₁-C₄-alkyl, cyano or C₁-C₄-alkoxy, such as chlorine, methyl, cyano, methoxy or ethoxy, especially chlorine or methyl, in particular chlorine.

A preferred embodiment of the compounds of the formula I relates to compounds of the formula I.1: 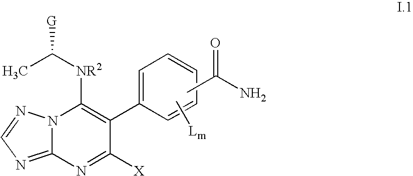
in which
G is C₂-C₆-alkyl, in particular ethyl, n- and isopropyl, n-, sec-, tert-butyl, and C₁-C₄-alkoxymethyl, in particular ethoxymethyl, or C₃-C₆-cycloalkyl, in particular cyclopentyl or cyclohexyl;
R² is hydrogen or methyl; and
X is chlorine, methyl, cyano, methoxy or ethoxy.

A further preferred embodiment of the compounds of the formula I relates to compounds in which R¹ and R² together with the nitrogen atom to which they are attached form a five- or six-membered heterocyclyl or heteroaryl which is attached via N and may contain a further heteroatom from the group consisting of O, N and S as ring member and/or one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkylenoxy. These compounds correspond in particular to formula I.2, 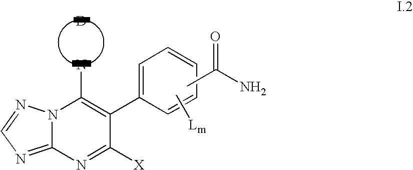
in which
D together with the nitrogen atom forms a five- or six-membered heterocyclyl or heteroaryl which is attached via N and may contain a further heteroatom from the group consisting of O, N and S as ring member and/or may carry one or more substituents from the group consisting of halogen, C₁-C₄-alkyl, C₁-C₄-alkoxy and C₁-C₂-haloalkyl; and
X is chlorine, methyl, cyano, methoxy or ethoxy.

A further preferred embodiment of the compounds of the formula I relates to compounds of the formula I.3 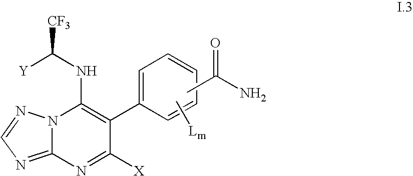
in which Y is hydrogen or C₁-C₄-alkyl, in particular methyl and ethyl, and X is chlorine, methyl, cyano, methoxy or ethoxy.

Particular preference is given to compounds of the formula I in which at least one substituent L is located ortho to the bond to the triazolopyrimidine skeleton, in particular a substituent selected from the group consisting of chlorine, fluorine and methyl.

A further preferred embodiment of the invention relates to compounds of the formula I in which the group C(O)NH₂ is located in the 4-position; the compounds correspond to formula I.A.

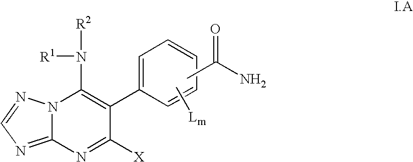

A further preferred embodiment of the invention relates to compounds of the formula I in which the group C(O)NH₂ is located in the 3-position; the compounds correspond to formula I.B.

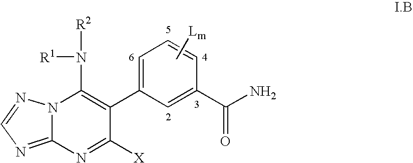

Particular preference is given to compounds of the formula I.A in which the phenyl group 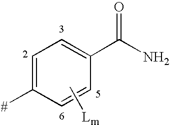
corresponds to group A: 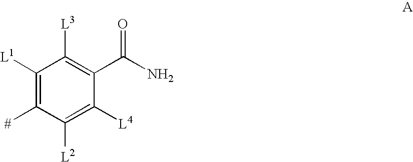
in which
L¹ is halogen, halomethyl or C₁-C₄-alkyl, in particular Cl, F or methyl;
L², L³, L⁴ are hydrogen or halogen, C₁-C₄-alkyl, C₁-C₂-haloalkyl or C₁-C₄-alkoxy, where # denotes the point of attachment to the triazolopyrimidine skeleton.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

Table 1

Compounds of the formula I.A in which X is chlorine, Lₘ is 2-chloro and the combination of R¹ and R² corresponds for each compound to one row of Table A

Table 2

Compounds of the formula I.A in which X is cyano, Lₘ is 2-chloro and the combination of R¹ and R² corresponds for each compound to one row of Table A

Table 3

Compounds of the formula I.A in which X is methyl, Lₘ is 2-chloro and the combination of R¹ and R² corresponds for each compound to one row of Table A

Table 4

Compounds of the formula I.A in which X is methoxy, Lₘ is 2-chloro and the combination of R¹ and R² corresponds for each compound to one row of Table A

Table 5

Compounds of the formula I.A in which X is chlorine, Lₘ is 2-fluoro and the combination of R¹ and R² corresponds for each compound to one row of Table A

Table 6

Compounds of the formula I.A in which X is cyano, Lₘ is 2-fluoro and the combination of R¹ and R² corresponds for each compound to one row of Table A

Table 7

Compounds of the formula I.A in which X is methyl, Lₘ is 2-fluoro and the combination of R¹ and R² corresponds for each compound to one row of Table A

Table 8

Compounds of the formula I.A in which X is methoxy, Lₘ is 2-fluoro and the combination of R¹ and R² corresponds for each compound to one row of Table A

Table 9

Compounds of the formula I.A in which X is chlorine, Lₘ is 2-chloro-6-fluoro and the combination of R¹ and R² corresponds for each compound to one row of Table A

Table 10

Compounds of the formula I.A in which X is cyano, Lₘ is 2-chloro-6-fluoro and the combination of R¹ and R² corresponds for each compound to one row of Table A

Table 11

Compounds of the formula I.A in which X is methyl, Lₘ is 2-chloro-6-fluoro and the combination of R¹ and R² corresponds for each compound to one row of Table A

Table 12

Compounds of the formula I.A in which X is methoxy, Lₘ is 2-chloro-6-fluoro and the combination of R¹ and R² corresponds for each compound to one row of Table A

Table 13

Compounds of the formula I.A in which X is chlorine, Lₘ is 2,6-dichloro and the combination of R¹ and R² corresponds for each compound to one row of Table A

Table 14

Compounds of the formula I.A in which X is cyano, Lₘ is 2,6-dichloro and the combination of R¹ and R² corresponds for each compound to one row of Table A

Table 15

Compounds of the formula I.A in which X is methyl, Lₘ is 2,6-dichloro and the combination of R¹ and R² corresponds for each compound to one row of Table A

Table 16

Compounds of the formula I.A in which X is methoxy, Lₘ is 2,6-dichloro and the combination of R¹ and R² corresponds for each compound to one row of Table A

Table 17

Compounds of the formula I.A in which X is chlorine, Lₘ is 2,6-difluoro and the combination of R¹ and R² corresponds for each compound to one row of Table A

Table 18

Compounds of the formula I.A in which X is cyano, Lₘ is 2,6-difluoro and the combination of R¹ and R² corresponds for each compound to one row of Table A

Table 19

Compounds of the formula I.A in which X is methyl, Lₘ is 2,6-difluoro and the combination of R¹ and R² corresponds for each compound to one row of Table A

Table 20

Compounds of the formula I.A in which X is methoxy, Lₘ is 2,6-difluoro and the combination of R¹ and R² corresponds for each compound to one row of Table A

Table 21

Compounds of the formula I.A in which X is chlorine, Lₘ is 2-methyl and the combination of R¹ and R² corresponds for each compound to one row of Table A

Table 22

Compounds of the formula I.A in which X is cyano, Lₘ is 2-methyl and the combination of R¹ ad R² corresponds for each compound to one row of Table A

Table 23

Compounds of the formula I.A in which X is methyl, Lₘ is 2-methyl and the combination of R¹ and R² corresponds for each compound to one row of Table A

Table 24

Compounds of the formula I.A in which X is methoxy, Lₘ is 2-methyl and the combination of R¹ and R² corresponds for each compound to one row of Table A

Table 25

Compounds of the formula I.A in which X is chlorine, Lₘ is 2-chloro, 5-fluoro and the combination of R¹ and R² corresponds for each compound to one row of Table A.

Table 26

Compounds of the formula I.A in which X is cyano, Lₘ is 2-chloro, 5-fluoro and the combination of R¹ and R² corresponds for each compound to one row of Table A

Table 27

Compounds of the formula I.A in which X is methyl, Lₘ is 2-chloro, 5-fluoro and the combination of R¹ and R² corresponds for each compound to one row of Table A

Table 28

Compounds of the formula I.A in which X is methoxy, Lₘ is 2-chloro, 5-fluoro and the combination of R¹ and R² corresponds for each compound to one row of Table A

Table 29

Compounds of the formula I.A in which X is chlorine, Lₘ is 2,5-difluoro and the combination of R¹ and R² corresponds for each compound to one row of Table A

Table 30

Compounds of the formula I.A in which X is cyano, Lₘ is 2,5-difluoro and the combination of R¹ and R² corresponds for each compound to one row of Table A

Table 31

Compounds of the formula I.A in which X is methyl, Lₘ is 2,5-difluoro and the combination of R¹ and R² corresponds for each compound to one row of Table A

Table 32

Compounds of the formula I.A in which X is methoxy, Lₘ is 2,5-difluoro and the combination of R¹ and R² corresponds for each compound to one row of Table A

Table 33

Compounds of the formula I.A in which X is chlorine, Lₘ is 2-chloro, 3,6-difluoro and the combination of R¹ and R² corresponds for each compound to one row of Table A

Table 34

Compounds of the formula I.A in which X is cyano, Lₘ is 2-chloro, 3,6-difluoro and the combination of R¹ and R² corresponds for each compound to one row of Table A

Table 35

Compounds of the formula I.A in which X is methyl, Lₘ is 2-chloro, 3,6-difluoro and the combination of R¹ and R² corresponds for each compound to one row of Table A

Table 36

Compounds of the formula I.A in which X is methoxy, Lₘ is 2-chloro, 3,6-difluoro and the combination of R¹ and R² corresponds for each compound to one row of Table A

Table 37

Compounds of the formula I.A in which X is chlorine, Lₘ is 2-chloro, 5,6-difluoro and the combination of R¹ and R² corresponds for each compound to one row of Table A

Table 38

Compounds of the formula I.A in which X is cyano, Lₘ is 2-chloro, 5,6-difluoro and the combination of R¹ and R² corresponds for each compound to one row of Table A

Table 39

Compounds of the formula I.A in which X is methyl, Lₘ is 2-chloro, 5,6-difluoro and the combination of R¹ and R² corresponds for each compound to one row of Table A

Table 40

Compounds of the formula I.A in which X is methoxy, Lₘ is 2-chloro, 5,6-difluoro and the combination of R¹ and R² corresponds for each compound to one row of Table A

Table 41

Compounds of the formula I.A in which X is chlorine, Lₘ is 2,6-dichloro, 5-fluoro and the combination of R¹ and R² corresponds for each compound to one row of Table A

Table 42

Compounds of the formula I.A in which X is cyano, Lₘ is 2,6-dichloro, 5-fluoro and the combination of R¹ and R² corresponds for each compound to one row of Table A

Table 43

Compounds of the formula I.A in which X is methyl, Lₘ is 2,6-dichlor, 5-fluoro and the combination of R¹ and R² corresponds for each compound to one row of Table A

Table 44

Compounds of the formula I.A in which X is methoxy, Lₘ is 2,6-dichloro, 5-fluoro and the combination of R¹ and R² corresponds for each compound to one row of Table A

Table 45

Compounds of the formula I.A in which X is chlorine, Lₘ is 2,3,6-trifluoro and the combination of R¹ and R² corresponds for each compound to one row of Table A

Table 46

Compounds of the formula I.A in which X is cyano, Lₘ is 2,3,6-trifluoro and the combination of R¹ and R² corresponds for each compound to one row of Table A

Table 47

Compounds of the formula I.A in which X is methyl, Lₘ is 2,3,6-trifluoro and the combination of R¹ and R³ corresponds for each compound to one row of Table A

Table 48

Compounds of the formula I.A in which X is methoxy, Lₘ is 2,3,6-trifluoro and the combination of R¹ and R² corresponds for each compound to one row of Table A

Table 49

Compounds of the formula I.A in which X is chlorine, Lₘ is 2-methyl, 5-fluoro and the combination of R¹ and R² corresponds for each compound to one row of Table A

Table 50

Compounds of the formula I.A in which X is cyano, Lₘ is 2-methyl, 5-fluoro and the combination of R¹ and R² corresponds for each compound to one row of Table A

Table 51

Compounds of the formula I.A in which X is methyl, Lₘ is 2-methyl, 5-fluoro and the combination of R¹ and R² corresponds for each compound to one row of Table A

Table 52

Compounds of the formula I.A in which X is methoxy, Lₘ is 2-methyl, 5-fluoro and the combination of R¹ and R² corresponds for each compound to one row of Table A

Table 53

Compounds of the formula I.A in which X is chlorine, Lₘ is 2,5-dichloro and the combination of R¹ and R² corresponds for each compound to one row of Table A

Table 54

Compounds of the formula I.A in which X is cyano, Lₘ is 2,5-dichloro and the combination of R¹ and R² corresponds for each compound to one row of Table A

Table 55

Compounds of the formula I.A in which X is methyl, Lₘ is 2,5-dichloro and the combination of R¹ and R² corresponds for each compound to one row of Table A

Table 56

Compounds of the formula I.A in which X is methoxy, Lₘ is 2,5-dichloro and the combination of R¹ and R² corresponds for each compound to one row of Table A

Table 57

Compounds of the formula I.A in which X is chlorine, Lₘ is 2-fluoro, 5-chloro and the combination of R¹ and R² corresponds for each compound to one row of Table A

Table 58

Compounds of the formula I.A in which X is cyano, Lₘ is 2-fluoro, 5-chloro and the combination of R¹ and R² corresponds for each compound to one row of Table A

Table 59

Compounds of the formula I.A in which X is methyl, Lₘ is 2-fluoro, 5-chloro and the combination of R¹ and R² corresponds for each compound to one row of Table A

Table 60

Compounds of the formula I.A in which X is methoxy, Lₘ is 2-fluoro, 5-chloro and the combination of R¹ and R² corresponds for each compound to one row of Table A

Table 61

Compounds of the formula I.A in which X is chlorine, Lₘ is 2,3-dichloro, 6-fluoro and the combination of R¹ and R² corresponds for each compound to one row of Table A

Table 62

Compounds of the formula I.A in which X is cyano, Lₘ is 2,3-dichloro, 6-fluoro and the combination of R¹ and R² corresponds for each compound to one row of Table A

Table 63

Compounds of the formula I.A in which X is methyl, Lₘ is 2,3-dichloro, 6-fluoro and the combination of R¹ and R² corresponds for each compound to one row of Table A

Table 64

Compounds of the formula I.A in which X is methoxy, Lₘ is 2,3-dichloro, 6-fluoro and the combination of R¹ and R² corresponds for each compound to one row of Table A

Table 65

Compounds of the formula I.A in which X is chlorine, Lₘ is 2,5-dichloro, 6-fluoro and the combination of R¹ and R² corresponds for each compound to one row of Table A

Table 66

Compounds of the formula I.A in which X is cyano, Lₘ is 2,5-dichloro, 6-fluoro and the combination of R¹ and R² corresponds for each compound to one row of Table A

Table 67

Compounds of the formula I.A in which X is methyl, Lₘ is 2,5-dichloro, 6-fluoro and the combination of R¹ and R² corresponds for each compound to one row of Table A

Table 68

Compounds of the formula I.A in which X is methoxy, Lₘ is 2,5-dichloro, 6-fluoro and the combination of R¹ and R² corresponds for each compound to one row of Table A

Table 69

Compounds of the formula I.A in which X is chlorine, Lₘ is 2,3,6-trichloro and the combination of R¹ and R² corresponds for each compound to one row of Table A

Table 70

Compounds of the formula I.A in which X is cyano, Lₘ is 2,3,6-trichloro and the combination of R¹ and R² corresponds for each compound to one row of Table A

Table 71

Compounds of the formula I.A in which X is methyl, Lₘ is 2,3,6-trichloro and the combination of R¹ and R² corresponds for each compound to one row of Table A

Table 72

Compounds of the formula I.A in which X is methoxy, Lₘ is 2,3,6-trichloro and the combination of R¹ and R² corresponds for each compound to one row of Table A

Table 73

Compounds of the formula I.A in which X is chlorine, Lₘ is 2,6-difluoro, 5-chloro and the combination of R¹ and R² corresponds for each compound to one row of Table A

Table 74

Compounds of the formula I.A in which X is cyano, Lₘ is 2,6-difluoro, 5-chloro and the combination of R¹ and R² corresponds for each compound to one row of Table A

Table 75

Compounds of the formula I.A in which X is methyl, Lₘ is 2,6-difluoro, 5-chloro and the combination of R¹ and R² corresponds for each compound to one row of Table A

Table 76

Compounds of the formula I.A in which X is methoxy, Lₘ is 2,6-difluoro, 5-chloro and the combination of R¹ and R² corresponds for each compound to one row of Table A

Table 77

Compounds of the formula I.A in which X is chlorine, Lₘ is 2-methyl, 5-chloro and the combination of R¹ and R² corresponds for each compound to one row of Table A

Table 78

Compounds of the formula I.A in which X is cyano, Lₘ is 2-methyl, 5-chloro and the combination of R¹ and R² corresponds for each compound to one row of Table A

Table 79

Compounds of the formula I.A in which X is methyl, Lₘ is 2-methyl, 5-chloro and the combination of R¹ and R² corresponds for each compound to one row of Table A

Table 80

Compounds of the formula I.A in which X is methoxy, Lₘ is 2-methyl, 5-chloro and the combination of R¹ and R² corresponds for each compound to one row of Table A

Table 81

Compounds of the formula I.A in which X is chlorine, Lₘ is 2-chloro, 5-methyl and the combination of R¹ and R² corresponds for each compound to one row of Table A

Table 82

Compounds of the formula I.A in which X is cyano, Lₘ is 2-chloro, 5-methyl and the combination of R¹ and R² corresponds for each compound to one row of Table A

Table 83

Compounds of the formula I.A in which X is methyl, Lₘ is 2-chloro, 5-methyl and the combination of R¹ and R² corresponds for each compound to one row of Table A

Table 84

Compounds of the formula I.A in which X is methoxy, Lₘ is 2-chloro, 5-methyl and the combination of R¹ and R² corresponds for each compound to one row of Table A

Table 85

Compounds of the formula I.A in which X is chlorine, Lₘ is 2-fluoro, 5-methyl and the combination of R¹ and R² corresponds for each compound to one row of Table A

Table 86

Compounds of the formula I.A in which X is cyano, Lₘ is 2-fluoro, 5-methyl and the combination of R¹ and R² corresponds for each compound to one row of Table A

Table 87

Compounds of the formula I.A in which X is methyl, Lₘ is 2-fluoro, 5-methyl and the combination of R¹ and R² corresponds for each compound to one row of Table A

Table 88

Compounds of the formula I.A in which X is methoxy, Lₘ is 2-fluoro, 5-methyl and the combination of R¹ and R² corresponds for each compound to one row of Table A

Table 89

Compounds of the formula I.A in which X is chlorine, Lₘ 2-chloro, 5-methyl, 6-fluoro and the combination of R¹ and R² corresponds for each compound to one row of Table A

Table 90

Compounds of the formula I.A in which X is cyano, Lₘ is 2-chloro, 5-methyl, 6-fluoro and the combination of R¹ and R² corresponds for each compound to one row of Table A

Table 91

Compounds of the formula I.A in which X is methyl, Lₘ is 2-chloro, 5-methyl, 6-fluoro and the combination of R¹ and R² corresponds for each compound to one row of Table A

Table 92

Compounds of the formula I.A in which X is methoxy, Lₘ is 2-chloro, 5-methyl, 6-fluoro and the combination of R¹ and R² corresponds for each compound to one row of Table A

Table 93

Compounds of the formula I.A in which X is chlorine, Lₘ is 2-chloro, 3-methyl, 6-fluoro and the combination of R¹ and R² corresponds for each compound to one row of Table A

Table 94

Compounds of the formula I.A in which X is cyano, Lₘ is 2-chloro, 3-methyl, 6-fluoro and the combination of R¹ and R² corresponds for each compound to one row of Table A

Table 95

Compounds of the formula I.A in which X is methyl, Lₘ is 2-chloro, 3-methyl, 6-fluoro and the combination of R¹ and R² corresponds for each compound to one row of Table A

Table 96

Compounds of the formula I.A in which X is methoxy, Lₘ is 2-chloro, 3-methyl, 6-fluoro and the combination of R¹ and R² corresponds for each compound to one row of Table A

Table 97

Compounds of the formula I.A in which X is chlorine, Lₘ is 2,6-dichloro, 5-methyl and the combination of R¹ and R² corresponds for each compound to one row of Table A

Table 98

Compounds of the formula I.A in which X is cyano, Lₘ is 2,6-dichloro, 5-methyl and the combination of R¹ and R² corresponds for each compound to one row of Table A

Table 99

Compounds of the formula I.A in which X is methyl, Lₘ is 2,6-dichloro, 5-methyl and the combination of R¹ and R² corresponds for each compound to one row of Table A

Table 100

Compounds of the formula I.A in which X is methoxy, Lₘ is 2,6-dichloro, 5-methyl and the combination of R¹ and R² corresponds for each compound to one row of Table A

Table 101

Compounds of the formula I.A in which X is chlorine, Lₘ is 2,6-difluoro, 3-methyl and the combination of R¹ and R² corresponds for each compound to one row of Table A

Table 102

Compounds of the formula I.A in which X is cyano, Lₘ is 2,6-difluoro, 3-methyl and the combination of R¹ and R² corresponds for each compound to one row of Table A

Table 103

Compounds of the formula I.A in which X is methyl, Lₘ is 2,6-difluoro, 3-methyl and the combination of R¹ and R² corresponds for each compound to one row of Table A

Table 104

Compounds of the formula I.A in which X is methoxy, Lₘ is 2,6-difluoro, 3-methyl and the combination of R¹ and R² corresponds for each compound to one row of Table A

Table 105

Compounds of the formula I.A in which X is chlorine, Lₘ is 2,5-dimethyl and the combination of R¹ and R² corresponds for each compound to one row of Table A

Table 106

Compounds of the formula I.A in which X is cyano, Lₘ is 2,5-dimethyl and the combination of R¹ and R² corresponds for each compound to one row of Table A

Table 107

Compounds of the formula I.A in which X is methyl, Lₘ is 2,5-dimethyl and the combination of R¹ and R² corresponds for each compound to one row of Table A

Table 108

Compounds of the formula I.A in which X is methoxy, Lₘ is 2,5-dimethyl and the combination of R¹ and R² corresponds for each compound to one row of Table A

Table 109

Compounds of the formula I.A in which X is chlorine, Lₘ is 2-chloro, 5-methoxy and the combination of R¹ and R² corresponds for each compound to one row of Table A

Table 110

Compounds of the formula I.A in which X is cyano, Lₘ is 2-chloro, 5-methoxy and the combination of R¹ and R² corresponds for each compound to one row of Table A

Table 111

Compounds of the formula I.A in which X is methyl, Lₘ is 2-chloro, 5-methoxy and the combination of R¹ and R² corresponds for each compound to one row of Table A

Table 112

Compounds of the formula I.A in which X is methoxy, Lₘ is 2-chloro, 5-methoxy and the combination of R¹ and R² corresponds for each compound to one row of Table A

Table 113

Compounds of the formula I.A in which X is chlorine, Lₘ is 2-fluoro, 5-methoxy and the combination of R¹ and R² corresponds for each compound to one row of Table A

Table 114

Compounds of the formula I.A in which X is cyano, Lₘ is 2-fluoro, 5-methoxy and the combination of R¹ and R² corresponds for each compound to one row of Table A

Table 115

Compounds of the formula I.A in which X is methyl, Lₘ is 2-fluoro, 5-methoxy and the combination of R¹ and R² corresponds for each compound to one row of Table A

Table 116

Compounds of the formula I.A in which X is methoxy, Lₘ is 2-fluoro, 5-methoxy and the combination of R¹ and R² corresponds for each compound to one row of Table A

Table 117

Compounds of the formula I.A in which X is chlorine, Lₘ is 2-chloro, 3-methoxy, 6-fluoro and the combination of R¹ and R² corresponds for each compound to one row of Table A

Table 118

Compounds of the formula I.A in which X is cyano, Lₘ is 2-chloro, 3-methoxy, 6-fluoro and the combination of R¹ and R² corresponds for each compound to one row of Table A

Table 119

Compounds of the formula I.A in which X is methyl, Lₘ is 2-chloro, 3-methoxy, 6-fluoro and the combination of R¹ and R² corresponds for each compound to one row of Table A

Table 120

Compounds of the formula I.A in which X is methoxy, Lₘ is 2-chloro, 3-methoxy, 6-fluoro and the combination of R¹ and R² corresponds for each compound to one row of Table A

Table 121

Compounds of the formula I.A in which X is chlorine, Lₘ is 2-chloro, 5-methoxy, 6-fluoro and the combination of R¹ and R² corresponds for each compound to one row of Table A

Table 122

Compounds of the formula I.A in which X is cyano, Lₘ is 2-chloro, 5-methoxy, 6-fluoro and the combination of R¹ and R² corresponds for each compound to one row of Table A

Table 123

Compounds of the formula I.A in which X is methyl, Lₘ is 2-chloro, 5-methoxy, 6-fluoro and the combination of R¹ and R² corresponds for each compound to one row of Table A

Table 124

Compounds of the formula I.A in which X is methoxy, Lₘ is 2-chloro, 5-methoxy, 6-fluoro and the combination of R¹ and R² corresponds for each compound to one row of Table A

Table 125

Compounds of the formula I.A in which X is chlorine, Lₘ is 2,6-dichloro, 5-methoxy and the combination of R¹ and R² corresponds for each compound to one row of Table A

Table 126

Compounds of the formula I.A in which X is cyano, Lₘ is 2,6-dichloro, 5-methoxy and the combination of R¹ and R² corresponds for each compound to one row of Table A

Table 127

Compounds of the formula I.A in which X is methyl, Lₘ is 2,6-dichloro, 5-methoxy and the combination of R¹ and R² corresponds for each compound to one row of Table A

Table 128

Compounds of the formula I.A in which X is methoxy, Lₘ is 2,6-dichloro, 5-methoxy and the combination of R¹ and R² corresponds for each compound to one row of Table A

Table 129

Compounds of the formula I.A in which X is chlorine, Lₘ is 2,6-difluoro, 3-methoxy and the combination of R¹ and R² corresponds for each compound to one row of Table A

Table 130

Compounds of the formula I.A in which X is cyano, Lₘ is 2,6-difluoro, 3-methoxy and the combination of R¹ and R² corresponds for each compound to one row of Table A

Table 131

Compounds of the formula I.A in which X is methyl, Lₘ is 2,6-difluoro, 3-methoxy and the combination of R¹ and R² corresponds for each compound to one row of Table A

Table 132

Compounds of the formula I.A in which X is methoxy, Lₘ is 2,6-difluoro, 3-methoxy and the combination of R¹ and R² corresponds for each compound to one row of Table A

Table 133

Compounds of the formula I.A in which X is chlorine, Lₘ is 2-methyl, 5-methoxy and the combination of R¹ and R² corresponds for each compound to one row of Table A

Table 134

Compounds of the formula I.A in which X is cyano, Lₘ is 2-methyl, 5-methoxy and the combination of R¹ and R² corresponds for each compound to one row of Table A

Table 135

Compounds of the formula I.A in which X is methyl, Lₘ is 2-methyl, 5-methoxy and the combination of R¹ and R² corresponds for each compound to one row of Table A

Table 136

Compounds of the formula I.A in which X is methoxy, Lₘ is 2-methyl, 5-methoxy and the combination of R¹ and R² corresponds for each compound to one row of Table A 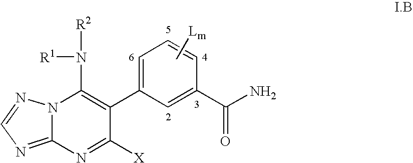
Table 137

Compounds of the formula I.B in which X is chlorine, Lₘ is 2-chloro and the combination of R¹ and R² corresponds for each compound to one row of Table A

Table 138

Compounds of the formula I.B in which X is cyano, Lₘ is 2-chloro and the combination of R¹ and R² corresponds for each compound to one row of Table A

Table 139

Compounds of the formula I.B in which X is methyl, Lₘ is 2-chloro and the combination of R¹ and R² corresponds for each compound to one row of Table A

Table 140

Compounds of the formula I.B in which X is methoxy, Lₘ is 2-chloro and the combination of R¹ and R² corresponds for each compound to one row of Table A

Table 141

Compounds of the formula I.B in which X is chlorine, Lₘ is 2-fluoro and the combination of R¹ and R² corresponds for each compound to one row of Table A

Table 142

Compounds of the formula I.B in which X is cyano, Lₘ is 2-fluoro and the combination of R¹ and R² corresponds for each compound to one row of Table A

Table 143

Compounds of the formula I.B, in which X is methyl, Lₘ is 2-fluoro and the combination of R¹ and R² corresponds for each compound to one row of Table A

Table 144

Compounds of the formula I.B, in which X is methoxy, Lₘ is 2-fluoro and the combination of R¹ and R² corresponds for each compound to one row of Table A

Table 145

Compounds of the formula I.B, in which X is chlorine, Lₘ is 2-chloro, 6-fluoro and the combination of R¹ and R² corresponds for each compound to one row of Table A

Table 146

Compounds of the formula I.B, in which X is cyano, Lₘ is 2-chloro, 6-fluoro and the combination of R¹ and R² corresponds for each compound to one row of Table A

Table 147

Compounds of the formula I.B, in which X is methyl, Lₘ is 2-chloro, 6-fluoro and the combination of R¹ and R² corresponds for each compound to one row of Table A

Table 148

Compounds of the formula I.B, in which X is methoxy, Lₘ 2-chloro, 6-fluoro and the combination of R¹ and R² corresponds for each compound to one row of Table A

Table 149

Compounds of the formula I.B, in which X is chlorine, Lₘ is 2,6-dichloro and the combination of R¹ and R² corresponds for each compound to one row of Table A

Table 150

Compounds of the formula I.B, in which X is cyano, Lₘ is 2,6-dichloro and the combination of R¹ and R² corresponds for each compound to one row of Table A

Table 151

Compounds of the formula I.B, in which X is methyl, Lₘ is 2,6-dichloro and the combination of R¹ and R² corresponds for each compound to one row of Table A

Table 152

Compounds of the formula I.B, in which X is methoxy, Lₘ is 2,6-dichloro and the combination of R¹ and R² corresponds for each compound to one row of Table A

Table 153

Compounds of the formula I.B, in which X is chlorine, Lₘ is 2,6-difluoro and the combination of R¹ and R² corresponds for each compound to one row of Table A

Table 154

Compounds of the formula I.B, in which X is cyano, Lₘ is 2,6-difluoro and the combination of R¹ and R² corresponds for each compound to one row of Table A

Table 155

Compounds of the formula I.B, in which X is methyl, Lₘ is 2,6-difluoro and the combination of R¹ and R² corresponds for each compound to one row of Table A

Table 156

Compounds of the formula I.B, in which X is methoxy, Lₘ is 2,6-difluoro and the combination of R¹ and R² corresponds for each compound to one row of Table A

Table 157

Compounds of the formula I.B, in which X is chlorine, Lₘ is 2-methyl and the combination of R¹ and R² corresponds for each compound to one row of Table A

Table 158

Compounds of the formula I.B, in which X is cyano, Lₘ is 2-methyl and the combination of R¹ and R² corresponds for each compound to one row of Table A

Table 159

Compounds of the formula I.B, in which X is methyl, Lₘ is 2-methyl and the combination of R¹ and R² corresponds for each compound to one row of Table A

Table 160

Compounds of the formula I.B, in which X is methoxy, Lₘ is 2-methyl and the combination of R¹ and R² corresponds for each compound to one row of Table A

Table 161

Compounds of the formula I.B, in which X is chlorine, Lₘ is 6-chloro and the combination of R¹ and R² corresponds for each compound to one row of Table A

Table 162

Compounds of the formula I.B, in which X is cyano, Lₘ is 6-chloro and the combination of R¹ and R² corresponds for each compound to one row of Table A

Table 163

Compounds of the formula I.B, in which X is methyl, Lₘ is 6-chloro and the combination of R¹ and R² corresponds for each compound to one row of Table A

Table 164

Compounds of the formula I.B, in which X is methoxy, Lₘ is 6-chloro and the combination of R¹ and R² corresponds for each compound to one row of Table A

Table 165

Compounds of the formula I.B, in which X is chlorine, Lₘ is 6-fluoro and the combination of R¹ and R² corresponds for each compound to one row of Table A

Table 166

Compounds of the formula I.B, in which X is cyano, Lₘ is 6-fluoro and the combination of R¹ and R² corresponds for each compound to one row of Table A

Table 167

Compounds of the formula I.B, in which X is methyl, Lₘ is 6-fluoro and the combination of R¹ and R² corresponds for each compound to one row of Table A

Table 168

Compounds of the formula I.B, in which X is methoxy, Lₘ is 6-fluoro and the combination of R¹ and R² corresponds for each compound to one row of Table A

Table 169

Compounds of the formula I.B, in which X is chlorine, Lₘ is 2-fluoro, 6-chloro and the combination of R¹ and R² corresponds for each compound to one row of Table A

Table 170

Compounds of the formula I.B, in which X is cyano, Lₘ is 2-fluoro, 6-chloro and the combination of R¹ and R² corresponds for each compound to one row of Table A

Table 171

Compounds of the formula I.B, in which X is methyl, Lₘ is 2-fluoro, 6-chloro and the combination of R¹ and R² corresponds for each compound to one row of Table A

Table 172

Compounds of the formula I.B, in which X is methoxy, Lₘ is 2-fluoro, 6-chloro and the combination of R¹ and R² corresponds for each compound to one row of Table A

Table 173

Compounds of the formula I.B, in which X is chlorine, Lₘ is 6-methyl and the combination of R¹ and R² corresponds for each compound to one row of Table A

Table 174

Compounds of the formula I.B, in which X is cyano, Lₘ is 6-methyl and the combination of R¹ and R² corresponds for each compound to one row of Table A

Table 175

Compounds of the formula I.B, in which X is methyl, Lₘ is 6-methyl and the combination of R¹ and R² corresponds for each compound to one row of Table A

Table 176

Compounds of the formula I.B, in which X is methoxy, Lₘ is 6-methyl and the combination of R¹ and R² corresponds for each compound to one row of Table A

TABLE A

| No. | R¹ | R² |
|---|---|---|
| A-1 | H | H |
| A-2 | CH₃ | H |
| A-3 | CH₃ | CH₃ |
| A-4 | CH₂CH₃ | H |
| A-5 | GH₂CH₃ | CH₃ |
| A-6 | CH₂CH₃ | CH₂CH₃ |
| A-7 | CH₂CF₃ | H |
| A-8 | CH₂CF₃ | CH₃ |
| A-9 | CH₂CF₃ | CH₂CH₃ |
| A-10 | CH₂CCl₃ | H |
| A-11 | CH₂CCl₃ | CH₃ |
| A-12 | CH₂CCl₃ | CH₂CH₃ |
| A-13 | CH₂CH₂CH₃ | H |
| A-14 | CH₂CH₂CH₃ | CH₃ |
| A-15 | CH₂CH₂CH₃ | CH₂CH₃ |
| A-16 | CH₂CH₂CH₃ | CH₂CH₂CH₃ |
| A-17 | CH(CH₃)₂ | H |
| A-18 | CH(CH₃)₂ | CH₃ |
| A-19 | CH(CH₃)₂ | CH₂CH₃ |
| A-20 | CH₂CH₂CH₂CH₃ | H |
| A-21 | CH₂CH₂CH₂CH₃ | CH₃ |
| A-22 | CH₂CH₂CH₂CH₃ | CH₂CH₃ |
| A-23 | CH₂CH₂CH₂CH₃ | CH₂CH₂CH₃ |
| A-24 | CH₂CH₂CH₂CH₃ | CH₂CH₂CH₂CH₃ |
| A-25 | (±) CH(CH₃)—CH₂CH₃ | H |
| A-26 | (±) CH(CH₃)—CH₂CH₃ | CH₃ |
| A-27 | (±) CH(CH₃)—CH₂CH₃ | CH₂CH₃ |
| A-28 | (S) CH(CH₃)—CH₂CH₃ | H |
| A-29 | (S) CH(CH₃)—CH₂CH₃ | CH₃ |
| A-30 | (S) CH(CH₃)—CH₂CH₃ | CH₂CH₃ |
| A-31 | (R) CH(CH₃)—CH₂CH₃ | H |
| A-32 | (R) CH(CH₃)—CH₂CH₃ | CH₃ |
| A-33 | (R) CH(CH₃)—CH₂CH₃ | CH₂CH₃ |
| A-34 | (±) CH(CH₃)—CH(CH₃)₂ | H |
| A-35 | (±) CH(CH₃)—CH(CH₃)₂ | CH₃ |
| A-36 | (±) CH(CH₃)—CH(CH₃)₂ | CH₂CH₃ |
| A-37 | (S) CH(CH₃)—CH(CH₃)₂ | H |
| A-38 | (S) CH(CH₃)—CH(CH₃)₂ | CH₃ |
| A-39 | (S) CH(CH₃)—CH(CH₃)₂ | CH₂CH₃ |
| A-40 | (R) CH(CH₃)—CH(CH₃)₂ | H |
| A-41 | (R) CH(CH₃)—CH(CH₃)₂ | CH₃ |
| A-42 | (R) CH(CH₃)—CH(CH₃)₂ | CH₂CH₃ |
| A-43 | (±) CH(CH₃)—C(CH₃)₃ | H |
| A-44 | (±) CH(CH₃)—C(CH₃)₃ | CH₃ |
| A-45 | (±) CH(CH₃)—C(CH₃)₃ | CH₂CH₃ |
| A-46 | (S) CH(CH₃)—C(CH₃)₃ | H |
| A-47 | (S) CH(CH₃)—C(CH₃)₃ | CH₃ |
| A-48 | (S) CH(CH₃)—C(CH₃)₃ | CH₂CH₃ |
| A-49 | (R) CH(CH₃)—C(CH₃)₃ | H |
| A-50 | (R) CH(CH₃)—C(CH₃)₃ | CH₃ |
| A-51 | (R) CH(CH₃)—C(CH₃)₃ | CH₂CH₃ |
| A-52 | (±) CH(CH₃)—CF₃ | H |
| A-53 | (±) CH(CH₃)—CF₃ | CH₃ |
| A-54 | (±) CH(CH₃)—CF₃ | CH₂CH₃ |
| A-55 | (S) CH(CH₃)—CF₃ | H |
| A-56 | S) CH(CH₃)—CF₃ | CH₃ |
| A-57 | (S) CH(CH₃)—CF₃ | CH₂CH₃ |
| A-58 | (R) CH(CH₃)—CF₃ | H |
| A-59 | (R) CH(CH₃)—CF₃ | CH₃ |
| A-60 | (R) CH(CH₃)—CF₃ | CH₂CH₃ |
| A-61 | (±) CH(CH₃)—CCl₃ | H |
| A-62 | (±) CH(CH₃)—CCl₃ | CH₃ |
| A-63 | (±) CH(CH₃)—CCl₃ | CH₂CH₃ |
| A-64 | (S) CH(CH₃)—CCl₃ | H |
| A-65 | (S) CH(CH₃)—CCl₃ | CH₃ |
| A-66 | (S) CH(CH₃)—CCl₃ | CH₂CH₃ |
| A-67 | (R) CH(CH₃)—CCl₃ | H |
| A-68 | (R) CH(CH₃)—CCl₃ | CH₃ |
| A-69 | (R) CH(CH₃)—CCl₃ | CH₂CH₃ |
| A-70 | CH₂CF₂CF₃ | H |
| A-71 | CH₂CF₂CF₃ | CH₃ |
| A-72 | CH₂CF₂CF₃ | CH₂CH₃ |
| A-73 | CH₂(CF₂)₂CF₃ | H |
| A-74 | CH₂(CF₂)₂CF₃ | CH₃ |
| A-75 | CH₂(CF₂)₂CF₃ | CH₂CH₃ |
| A-76 | CH₂C(CH₃)═CH₂ | H |
| A-77 | CH₂C(CH₃)═CH₂ | CH₃ |
| A-78 | CH₂C(CH₃)═CH₂ | CH₂CH₃ |
| A-79 | CH₂CH═CH₂ | H |
| A-80 | CH₂CH═CH₂ | CH₃ |
| A-81 | CH₂CH═CH₂ | CH₂CH₃ |
| A-82 | CH(CH₃)CH═CH₂ | H |
| A-83 | CH(CH₃)CH═CH₂ | CH₃ |
| A-84 | CH(CH₃)CH═CH₂ | CH₂CH₃ |
| A-85 | CH(CH₃)C(CH₃)═CH₂ | H |
| A-86 | CH(CH₃)C(CH₃)═CH₂ | CH₃ |
| A-87 | CH(CH₃)C(CH₃)═CH₂ | CH₂CH₃ |
| A-88 | CH₂—C≡CH | H |
| A-89 | CH₂—C≡CH | CH₃ |
| A-90 | CH₂—C≡CH | CH₂CH₃ |
| A-91 | cyclopentyl | H |
| A-92 | cyclopentyl | CH₃ |
| A-93 | cyclopentyl | CH₂CH₃ |
| A-94 | cyclohexyl | H |
| A-95 | cyclohexyl | CH₃ |
| A-96 | cyclohexyl | CH₂CH₃ |
| A-97 | CH₂—C₆H₅ | H |
| A-98 | CH₂—C₆H₅ | CH₃ |
| A-99 | CH₂—C₆H₅ | CH₂CH₃ |

| | |
|---|---|
| A-100 | —(CH₂)₂CH═CHCH₂— |
| A-101 | —(CH₂)₂C(CH₃)═CHCH₂— |
| A-102 | —CH(CH₃)CH₂—CH═CHCH₂— |
| A-103 | —(CH₂)₂CH(CH₃)(CH₂)₂— |
| A-104 | —(CH₂)₃CHFCH₂— |
| A-105 | —(CH₂)₂CHF(CH₂)₂— |
| A-106 | —CH₂CHF(CH₂)₃— |
| A-107 | —(CH₂)₂CH(CF₃)(CH₂)₂— |
| A-108 | —(CH₂)₂O(CH₂)₂— |
| A-109 | —(CH₂)₂S(CH₂)₂— |
| A-110 | —(CH₂)₅— |
| A-111 | —(CH₂)₄— |
| A-112 | —CH₂CH═CHCH₂— |
| A-113 | —CH(CH₃)(CH₂)₃— |
| A-114 | —CH₂CH(CH₃)(CH₂)₂— |
| A-115 | —CH(CH₃)—(CH₂)₂—CH(CH₃)— |
| A-116 | —CH(CH₃)—(CH₂)₄— |
| A-117 | —CH₂—CH(CH₃)—(CH₂)₃— |
| A-118 | —(CH₂)—CH(CH₃)—CH₂—CH(CH₃)—CH₂— |
| A-119 | —CH(CH₂CH₃)—(CH₂)₄— |
| A-120 | —(CH₂)₂—CHOH—(CH₂)₂— |
| A-121 | —(CH₂)₆— |
| A-122 | —CH(CH₃)—(CH₂)₅— |
| A-123 | —(CH₂)₂—N(CH₃)—(CH₂)₂— |
| A-124 | —N═CH—CH═CH— |
| A-125 | —N═C(CH₃)—CH═C(CH₃)— |
| A-126 | —N═C(CF₃)—CH═C(CF₃)— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septodia tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg of seed, preferably 1 to 200 g/100 kg, in particular 5 to 100 g/100 kg are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water
A Water-soluble concentrates (SL)

10 parts by weight of a compound according to the invention are dissolved in water or in a water-soluble solvent. As an alternative, wetters or other auxiliaries are added. The active compound dissolves upon dilution with water.
B Dispersible concentrates (DC)

20 parts by weight of a compound according to the invention are dissolved in cyclohexanone with addition of a dispersant, for example polyvinylpyrrolidone. Dilution with water gives a dispersion.
C Emulsifiable concentrates (EC)

15 parts by weight of a compound according to the invention are dissolved in xylene with addition of calcium dodecylbenzenesulfonate and castor oil ethoxylate (in each case 5%). Dilution with water gives an emulsion.
D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.
E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.
F Water-dispersible granules and water-soluble granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.
G Water-dispersible powders and water-soluble powders (WP, SP)

75 parts by weight of a compound according to the invention are ground in a rotor-stator mill with addition of dispersants, wetters and silica gel. Dilution with water gives a stable dispersion or solution of the active compound.

2. Products to be Applied Undiluted

H Dustable powders (DP)

5 parts by weight of a compound according to the invention are ground finely and mixed intimately with 95% of finely divided kaolin. This gives a dustable product.
I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.
J ULV solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, flutriafol, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds by appropriate modification of the starting compounds. The resulting compounds are listed in the subsequent table with physical data.

### EXAMPLE 1

### Preparation of 5-chloro-6-(2,6-difluoro-4-aminocarbonylphenyl)-7-(4-methylpiperidin-1-yl)-[1,2,4]-triazolo[1,5-a]pyrimidine

HCl gas was passed through a solution of 1.5 mmol of 5-chloro-6-(2,6-difluoro-4-cyanophenyl)-7-(4-methylpiperidin-1-yl)-[1,2,4]-triazolo[1,5-a]pyrimidine [cf. WO 03/80615] in 20 ml of methanol until saturation was achieved. After 24 hours of stirring at about 20-25° C., the precipitate that had formed was filtered off and dissolved in dichloromethane. The solution was washed with water and dried, and the solvent was then removed. Chromatography on silica gel gave 140 mg of the title compound of m.p. 197° C. and 200 mg of the corresponding methyl ester of m.p. 176° C.

### EXAMPLE 2

### Preparation of 5-methyl-6-(2,6-difluoro-4-aminocarbonylphenyl)-7-(4-methylpiperidinyl)-1,2,4-triazolo[1,5a]pyrimidine

### Step 2a: 5-(dimethylmalon-2-yl)-6-(2,6-difluoro-4-cyanophenyl)-7-(4-methylpiperidinyl)-1,2,4-triazolo[1,5a]pyrimidine

A solution of 1 g (2.7 mmol) of 5-chloro-6-(2,6-difluoro-4-cyanophenyl)-7-(4-methylpiperidinyl)-1,2,4-triazolo[1,5a]pyrimidine [cf. WO 03/80615] and 1 g of sodium dimethylmalonate (6.5 mmol) in 10 ml of acetonitrile was stirred at 70-80° C. for 4 hours and at 20-25° C. for 2.5 days. During this time, a yellow solid precipitate formed. The reaction mixture was filtered off through kieselguhr and the precipitate was stirred in a mixture of dil. hydrochloric acid and methylene chloride until it was decolorized. After phase separation, the aqueous phase was extracted with methylene chloride. Drying of the combined organic phases and distillative removal of the solvent gave 1 g of the title compound.

¹H-NMR (CDCl₃, δ in ppm): 8.4 (s, 1H); 7.4 (d, 2H); 4.65 (s, 1H); 3.7/s, 6H); 3.55 (d, broad, 2H); 3.8 (t, broad, 2H); 1.65 (d, broad, 2H); 1.55 (m, 1H); 1.3 (m, 2H); 0.95 (d, 3H).

### Step 2b: 5-methyl-6-(2,6-difluoro-4-aminocarbonylphenyl)-7-(4-methylpiperidinyl)-1,2,4-triazolo[1,5a]pyrimidine

A solution of 0.5 g (1.03 mmol) of 5-(dimethylmalon-2-yl)-6-(2,6-difluoro-4-cyanophenyl)-7-(4-methylpiperidinyl)-1,2,4-triazolo[1,5a]pyrimidine and 0.3 g (3.8 mmol) of 50% strength aqueous sodium hydroxide solution in 6 ml of methanol/water (5:1) was heated under reflux for 3 hours. After dilution with methyl t-butyl ether (MTBE) and dilute hydrochloric acid, the organic phase was separated off and the aqueous phase was extracted with MTBE. The combined organic phases were dried and freed from the solvent. The residue was purified by preparative MPLC on silica gel RP-18 using acetonitrile/water mixtures. The eluate gave, after removal of the solvent, 0.11 g of the title compound as a yellow resin.

¹H-NMR (CDCl₃, δ in ppm): 8.35 (s, 1H); 7.8 (d, 2H); 7.7 (s, broad, 1H); 6.75 (s, broad, 1H); 3.65 (d, 2H); 2.8 (t, 2H); 2.35 (s, 3H); 1.6 (d, 2H); 1.5 (m, 1H); 1.3 (m, 2H); 0.9 (d, 3H).

TABLE 1
Compounds of the formula I

| | |
|---|---|
| | I |
| 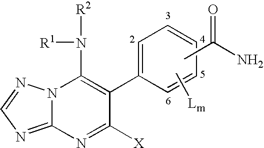 | |

| | | | | | position | phys. Data (m.p. [° C.]; |
|---|---|---|---|---|---|---|
| No. | R¹ | R² | X | Lₘ | CONH₂ | ¹H-NMR δ [ppm]) |
| I-1 | —(CH₂)₂—CH(CH₃)—(CH₂)₂— | | Cl | 2,6-F₂ | 4 | 197 |
| I-2 | —(CH₂)₂—CH(CH₃)—(CH₂)₂— | | CH₃ | 2,6-F₂ | 4 | (Ex. 2b) |
| I-3 | —(CH₂)₂—CH(CH₃)—(CH₂)₂— | | Cl | 2-F | 4 | 8.4 (s); 7.7 (m); |
| | | | | | | 7.4 (t); 0.95 (d) |
| I-4 | —(CH₂)₂—CH(CH₃)—(CH₂)₂— | | Cl | 2,5-(CH₃)₂ | 4 | 130 |
| I-5 | —(CH₂)₂—CH(CH₃)—(CH₂)₂— | | Cl | 2-CH₃ | 4 | 162-164 |
| I-6 | (R) | H | Cl | 2,5-(CH₃)₂ | 4 | 240-241 |
| | CH(CH₃)C(CH₃)₃ | | | | | |
| I-7 | (R) | H | Cl | 2-CH₃ | 4 | 184 |
| | CH(CH₃)C(CH₃)₃ | | | | | |
| I-8 | —(CH₂)₂—CH(CH₃)—(CH₂)₂— | | Cl | 2-Cl,5-OCH₃ | 4 | 190-191 |
| I-9 | —(CH₂)₂—CH(CH₃)—(CH₂)₂— | | Cl | 2,5-Cl₂ | 4 | 198-199 |
| I-10 | —(CH₂)₂—CH(CH₃)—(CH₂)₂— | | Cl | 2-CH₃,5-F | 4 | 215-216 |
| I-11 | (R) | H | Cl | 2-CH₃,5-F | 4 | 214-215 |
| | CH(CH₃)C(CH₃)₃ | | | | | |
| I-12 | —(CH₂)₂—CH(CH₃)-(CH₂)_{2—} | | Cl | 2-CH₃,5-Cl | 4 | 223-225 |
| I-13 | (R) | H | Cl | 2-CH₃, | 4 | 2 atropisomers: |
| | CH(CH₃)C(CH₃)₃ | | | 5-OCH₃ | | 8.45; 8.4 (2s, 1H); |
| | | | | | | 8.3; 8.35 (2s, 1H); |
| | | | | | | 7.25; 7.1 (2s, 1H); |
| | | | | | | 1.1; 1.0 (2d, 3H) |
| I-14 | —(CH₂)₂—CH(CH₃)—(CH₂)₂— | | Cl | 2—Cl | 4 | 247-249 |
| I-15 | (R) | H | Cl | 2-Cl | 4 | 2 atropisomers: |
| | CH(CH₃)C(CH₃)₃ | | | | | 8.35 (s, 1H); 8.15 (d, |
| | | | | | | 1H); 7.95 (m, 1H); |
| | | | | | | 7.55; 7.5 (2d, 1H); |
| | | | | | | 1.05; 0.9 (2d, 3H) |
| I-16 | (R) | H | Cl | 2-CH₃, 5-Cl | 4 | 2 atropisomers: |
| | CH(CH₃)C(CH₃)₃ | | | | | 8.35 (s, 1H); 7.95; 7.9 |
| | | | | | | (2s, 1H); 7.4; 7.35 |
| | | | | | | (2s, 1H); 1.0 (d, 3H) |
| I-17 | —(CH₂)₂—CH(CH₃)—(CH₂)_{2—} | | Cl | 2-Cl,5-CH₃ | 4 | 205-208 |
| I-18 | (R) | H | Cl | 2-Cl,5-CH₃ | 4 | >250 |
| | CH(CH₃)C(CH₃)₃ | | | | | |
| I-19 | (R) | H | Cl | 2-CH₃ | 5 | >250 |
| | CH(CH₃)C(CH₃)₃ | | | | | |
| I-20 | —(CH₂)₂—CH(CH₃)—(CH₂)_{2—} | | Cl | 2-Cl | 5 | 8.3 (s); 8.0 (s); |
| | | | | | | 7.9 (d); 7.5 (d); 0.9 (d) |
| I-21 | —(CH₂)₂—CH(CH₃)—(CH₂)_{2—} | | Cl | 2-CH₃ | 5 | 223 |
| I-22 | —(CH₂)₂—CH(CH₃)—(CH₂)_{2—} | | Cl | 2-CH₃ | 5 | 246-247 |
| | | | | 4-OCH₃ | | |
| I-23 | (R) | H | Cl | 2-Cl | 5 | 2 atropisomers: 8.4; |
| | CH(CH₃)C(CH₃)₃ | | | | | 8.3 (2s, 1H); 8.2 (2s, |
| | | | | | | 1H); 8.1; 8.03 (2d, |
| | | | | | | 1H); 7.65 (d, 1H); |
| | | | | | | 1.05; 0.95 (2d, 3H) |
| I-24 | (R) | H | Cl | 2-CH₃, | 5 | Polar (HPLC) |
| | CH(CH₃)C(CH₃)₃ | | | 4-OCH₃ | | atropisomer: 8.35 (s, |
| | | | | | | 1H); 8.1 (s, 1H); 7.0 |
| | | | | | | (s, 1H); 0.95 (d, 3H) |
| I-25 | (R) | H | Cl | 2-CH₃, | 5 | Unpolar (HPLC) |
| | CH(CH₃)C(CH₃)₃ | | | 4-OCH₃ | | atropisomer: 8.35 (s, |
| | | | | | | 1H); 8.15 (s, 1H); 7.0 |
| | | | | | | (s, 1H); 0.95 (d, 3H) |

Examples for the Action Against Harmful Fungi

The fungicidal action of the compounds of the formula I was demonstrated by the following tests:

The active compounds were prepared as a stock solution with 0.25% by weight of active compound in acetone or DMSO. 1% by weight of the emulsifier Uniperol® EL (wetting agent having emulsifying and dispersing action based on ethoxylated alkylphenols) was added to this solution, and the mixture was diluted with water to the desired concentration.

### Use Example 1—Activity Against Net Blotch of Barley Caused by Pyrenophora teres

Leaves of pot-grown barley seedlings of the cultivar “Hanna” were sprayed to runoff point with an aqueous suspensiori having the concentration of active compound stated below. 24 hours after the spray coating had dried on, the test plants were inoculated with an aqueous spore suspension of Pyrenophora [syn. Drechslera] teres, the causative agent of net blotch. The test plants were subsequently placed in a greenhouse at temperatures of between 20 and 24° C. and at a relative atmospheric humidity of 95 to 100%. After 6 days, the extent of the development of the disease was determined visually in % of infection of the total leaf area.

In this test, the plants which had been treated with 63 ppm of the compounds I-1 or I-25 showed an infection of only about 3%, whereas the untreated plants were 90% infected.

### Use Example 2—Activity Against Peronospora of Vines Caused by Plasmopara viticola

Leaves of potted vines of the cultivar “Riesling” were sprayed to runoff point with an aqueous suspension having the concentration of active compounds stated below. The next day, the undersides of the leaves were inoculated with an aqueous zoospore suspension of Plasmopara viticola. The vines were then initially placed in a water-vapor-saturated chamber at 24° C. for 48 hours and then in a greenhouse at temperatures of between 20 and 30° C. for 5 days. After this time, the plants were again placed in a moist chamber for 16 hours to promote sporangiophore eruption. The extent of the development of the infection on the undersides of the leaves was then determined visually.

In this test, the plants which had been treated with 250 ppm of the title compound from example 1 showed no infection, whereas the untreated plants were 90% infected.

### Use Example 3—Activity Against Gray Mold on Bell-pepper Leaves Caused by Botrytis cinerea, Protective Application

Bell-pepper seedlings of the cultivar “Neusiedler Ideal Elite ” were, after 2 to 3 leaves were well developed, sprayed to run-off point with an aqueous suspension having the concentration of active compounds stated below. The next day, the treated plants were inoculated with a spore suspension of Botrytis cinerea which contained 1.7×10⁶ spores/ml in a 2% aqueous biomold solution. Test plants were then placed in a dark climatized chamber at 22 to 24° C. and high atmospheric humidity. After 5 days, the extent of the fungal infection on the leaves could be determined visually in %.

In this test, the plants which had been treated with 63 ppm of the compounds I-2, I-14, I-23, I-24 or I-25 showed an infection of not more than 30%, whereas the untreated plants were 75% infected.

1. A triazolopyrimidine of the formula I 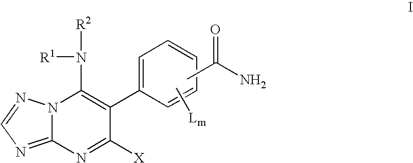
in which the substituents are as defined below:
R¹, R² independently of one another are hydrogen, C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl or a five- or six-membered saturated, partially unsaturated or aromatic heterocycle which contains one to four heteroatoms from the group consisting of O, N and S,
R¹ and R² together with the nitrogen atom to which they are attached may also form a five- or six-membered heterocyclyl or heteroaryl which is attached via N and may contain one to three further heteroatoms from the group consisting of O, N and S as ring members and/or may carry one or more substituents from the group consising of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
R¹ and/or R² may carry one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkylenoxy, phenyl, naphthyl, a five- to ten-membered saturated, partially unsaturated or aromatic heterocycle which contains one to four heteroatoms from the group consisting of O, N and S,
where these aliphatic, alicyclic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the abovementioned alkenyl or alkynyl groups in these radicals contain 2 to 8 carbon atoms;
and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups;
L is halogen, cyano, C₁-C₆-alkyl, C₁-C₄-haloalkyl, C₁-C₆-alkoxy, C₃-C₆-alkenyloxy or C₁-C₄-alkoxycarbonyl;
m is 1, 2, 3 or 4, where the groups L may be different if m is greater than 1;
X is halogen, cyano, C₁-C₄-alkyl, C₁-C₄-haloalkyl, C₁-C₄-alkoxy or C₁-C₂-haloalkoxy.

2. The compound of the formula I as claimed in claim 1, in which R¹ is not hydrogen.

3. A compound of the formula I.A: 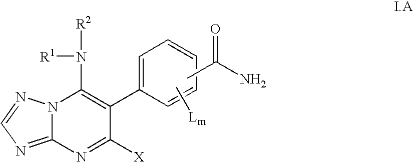
in which the variables are as defined in claim 1.

4. The compound of the formula I.A as claimed in claim 3 in which the phenyl group 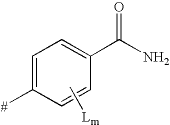
corresponds to the group A: 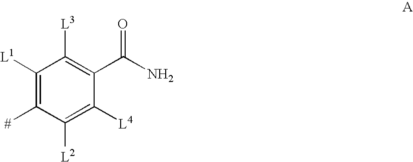
where
L¹ is halogen, halomethyl or C₁-C₄-alkyl;
L₂, L³, L⁴ are hydrogen or halogen, C₁-C₄-alkyl, C₁-C₂-haloalkyl or C₁-C₄-alkoxy.

5. A process for preparing the compounds of the formula I as claimed in claim 1 in which X is halogen, cyano, C₁-C₄-alkyl, C₁-C₄-alkoxy or C₁-C₂-haloalkoxy, by reaction of 5-aminotriazole of the formula II 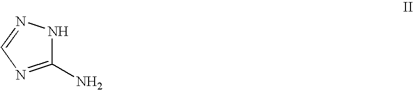
with phenymalonates of the formula III, 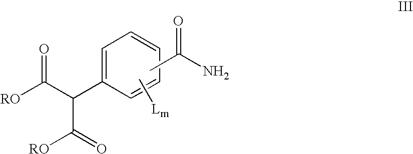
in which R is alkyl, with dihydroxytriazolopyrimidines of the formula IV, 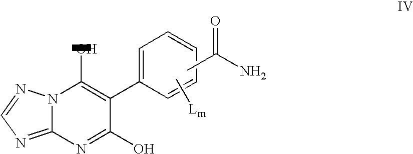
halogenation to give the dihalo compounds of the formula V 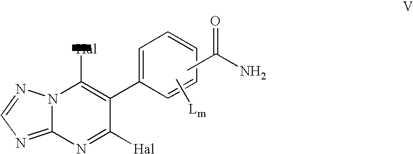
and reaction of V with amines of the formula VI 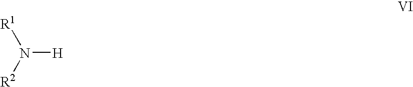
to give compounds of the formula I in which X is halogen, if desired, to prepare compounds I in which X is cyano, C₁-C₄-alkoxy or C₁-C₂-haloalkoxy, reaction of compounds I in which X is halogen with compounds of the formula VII,
M-X′ VII
which, depending on the group X′ to be introduced, are inorganic cyanides, alkoxides or haloalkoxides and in which M is an ammonium, tetraalkylammonium, alkali metal or alkaline earth metal cation, and, if desired, to prepare compounds of the formula I as claimed in claim 1 in which X is alkyl, by reaction of the compounds I in which X is halogen with malonates of the formula VIII, 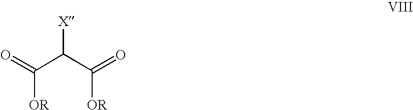
in which X″ is hydrogen or C₁-C₃-alkyl and R is C₁-C₄-alkyl, to give compounds of the formula IX 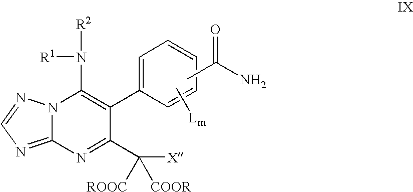
and decarboxylation to give compounds I in which X is alkyl.

6. A process for preparing the compounds of the formula I as claimed in claim 1 in which X is C₁-C₄-alkyl or C₁-C₄-haloalkyl, by reaction of 5-aminotriazole of the formula II as set forth in claim 5 with keto esters of the formula IIIa, 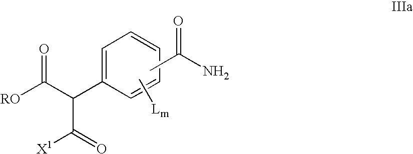
in which X¹ is C₁-C₄-alkyl or C₁-C₄-haloalkyl and R is C₁-C₄-alkyl, to give 5-alkyl-7-hydroxy-6-phenyltriazolopyrimidines of the formula IVa 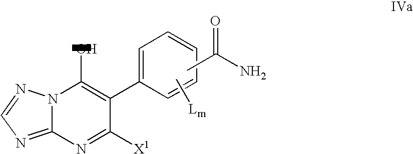
halogenation of IVa to give 7-halotriazolopyrimidines of the formula Va 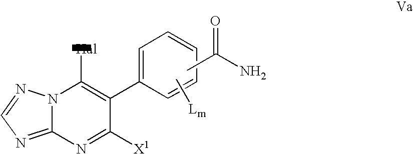
and reaction of Va with amines of the formula VI as set forth in claim 5 to give compounds I in which X is C₁-C₄-alkyl or C₁-C₄-haloalkyl.

7. A compound of the formula IV, IVa, V or Va as set forth in claim 5.

8. A process for preparing compounds of the formula I as claimed in claim 1 by reacting 6-cyanophenyltriazolopyrimidines of the formula XI 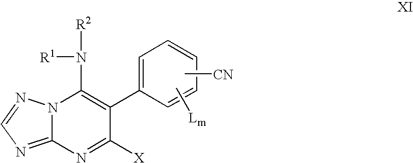
in the presence of sulfuric acid or in a polyethylene glycol/NaOH system or with urea/hydrogen peroxide.

9. A fungicidal composition, comprising a solid or liquid carrier and a compound of the formula I as claimed in claim 1.

10. Seed, comprising 1 to 1000 g of a compound of the formula I as claimed in claim 1 per 100 kg.

11. A method for controlling phytopathogenic harmful fungi, which method comprises treating the fungi or the materials, plants, the soil or seed to be protected against fungal attach with an effective amount of a compound of the formula I as claimed in claim 1.

12. A compound of the formula IV, IVa, V or Va as set forth in claim 6.
